(19) United States
(12) Patent Application Publication
(10) Pub. No.: US 2021/0154472 A1
(43) Pub. Date: May 27, 2021

(54) ELECTRICALLY RESPONSIVE, NANOPATTERNED SURFACE FOR TRIGGERED INTRACELLULAR DELIVERY OF BIOLOGICALLY ACTIVE MOLECULES

(71) Applicants: Yissum Research Development Company of the Hebrew University of Jerusalem Ltd., Jerusalem (IL); ACADEMIA SINICA, Taipei (TW)

(72) Inventors: Roy SHENHAR, Mevasseret Zion (IL); Meital RECHES, Kfar Bin Nun (IL); Hsiao-hua YU, Taipei (TW)

(21) Appl. No.: 17/105,056

(22) Filed: Nov. 25, 2020

Related U.S. Application Data
(60) Provisional application No. 62/941,222, filed on Nov. 27, 2019.

(51) Int. Cl. A61N 1/32; A61K 9/00; B32B 27/08; C08G 61/12; A61K 9/70; B32B 27/28

(52) U.S. Cl. CPC A61N 1/327; A61K 9/0009; B32B 27/08; B82Y 5/00; A61K 9/7007; B32B 27/28; C08G 61/126

(57) ABSTRACT

Nano-patterned devices for triggered intracellular delivery of active materials are disclosed. The device may comprise a nano-sized polyelectrolyte multilayer (PEM) comprising at least one layer of an electroactive polyelectrolyte polymer, where the PEM is configured to hold or receive an active material to be disposed within the multilayer and to release the active material under an electric field.

### CROSS-REFERENCE TO RELATED APPLICATIONS

This application claims the benefit of U.S. Provisional Application No. 62/941,222, filed Nov. 27, 2019, which is hereby incorporated by reference in its entirety.

### TECHNOLOGICAL FIELD

The invention generally contemplates a nano-patterned device for triggered intracellular delivery of active materials.

### BACKGROUND OF THE INVENTION

Efficient delivery of biologically active ingredients, ranging from growth factors, therapeutic molecules to genetic matter holds the key to program cell functions and viability. In order to achieve efficient delivery, polymer thin films and coatings were among the most successful and promising tools. They have been applied to promote surface mediated delivery for drug-eluting implants/stents, cell reprogramming for regenerative medicine and controlled release of active therapeutic ingredients.

Considering all the engineering and biological aspects required, polyelectrolyte multilayer (PEM) films built by layer-by-layer techniques have emerged due to their simplicity, the use of tunable material compositions and stability for ingredient storage and controlled release. When PEMs were applied for programming cell functions/viability, they demonstrated sustained release of encapsulated ingredients from the surface-initiated decomposition at the cells/materials interface. For cellular applications, it would be critical to integrate PEM techniques with other advanced material features in order to achieve higher delivery efficiency, larger active-ingredient loading, better stability in aqueous solutions, and different release profiles.

Recently [1], it was demonstrated that the introduction of nano-structural features into materials provided spatial-temporal control over cell functions. Notably, specific cell-substrate interactions observed on three-dimensional (3D) micro/nanostructures provided the topographic cues regulating the cell spreading morphology, thereby promoting the level of cell differentiation for stem cells, enhancing the transfection efficiency of cells with targeted gene expression and improving the capturing efficiency of circulating tumor cells (CTCs) for noninvasive blood biopsies.

Among the various approaches used for fabrication of nano-patterned substrates, block copolymers (BCPs) provide a simple, economic and versatile platform. Owing to their microphase separation, block copolymer materials are inherently structured and exhibit periodic morphologies such as alternating lamellae, hexagonally-packed cylinders (made of a short block surrounded by a matrix consisting of a long block), and spheres in a matrix. The typical periodicity of these structures is in the range of a few nanometers to several hundreds of nanometers, dictated by the total length of the copolymer. In thin films, such morphologies translate to surface patterns, where standing lamellae (i.e., lamellae oriented normal to the substrate) and lying cylinders give rise to striped patterns, where standing cylinders and spheres lead to dotted patterns.

Shenhar et al [2-5] have demonstrated the utilization of surface patterns made of polystyrene-block-poly (2-vinyl pyridine) (PS-b-P2VP) for the assembly of nano-patterned PEMs. In this approach, P2VP forms domains that are cross-linked after the formation of the surface pattern by reaction of the pyridine units with 1,4-diiodobutane (DIB), which also quaternizes the pyridines. This treatment renders the cross-linked P2VP domains (denoted as “xP2VP” hereafter) positively charged and hence amenable for layer-by-layer assembly of polyelectrolytes. The polymer film hence presents alternating neutral and positively charged domains (the PS and xP2VP domains) and can thus serve as a template for the preparation of nano-patterned PEMs.

Hammond et al [6-8] have demonstrated fabrication of nanoscale electroactive thin films that can be engineered to undergo controlled dissolution in the presence of a small applied voltage to release chemical agents.

Dong et al [9] have demonstrated selective electrodeposition o inorganic ions and DNA multilayer film for tunable release of DNA.

Su et al [10] have disclosed electric-stimulus-responsive multilayer films based on a cobaltocenium-containing polymer.

### BACKGROUND PUBLICATIONS

[1] Ito, Y.; Hoare, M.; Narita, M. Spatial and Temporal Control of Senescence. Trends Cell Biol. 2017, 27, 820-832.
[2] Asor, L.; Nir, S.; Oded, M.; Reches, M.; Shenhar, R. Nano-patterned polyelectrolyte multilayers assembled using block copolymer templates: The combined effect of ionic strength and nano-confinement. Polymer 2017, 126, 56-64.
[3] Oded, M.; Kelly, S. T.; Gilles, M. K.; Müller, A. H. E.; Shenhar, R. From dots to doughnuts: Two-dimensionally confined deposition of polyelectrolytes on block copolymer templates. Polymer 2016, 107, 406-414.
[4] Oded, M.; Müller, A. H. E.; Shenhar, R. A block copolymer-templated construction approach for the creation of nano-patterned polyelectrolyte multilayers and nanoscale objects. Soft Matter 2016, 12, 8098-8103.
[5] Oded, M.; Kelly, S. T.; Gilles, M. K.; Müller, A. H. E.; Shenhar, R. Periodic nanoscale patterning of polyelectrolytes over square centimeter areas using block copolymer templates. Soft Matter 2016, 12, 4595-4602.
[6] Wood, K. C.; Zacharia, N. S.; Schmidt, D. J.; Wrightman, S. N.; Andaya, B. J.; Hammond, P. T. “Electroactive controlled release thin films”, Proc. Nat. Acad. Sci. U.S.A. 2008, 105, 2280-2285.
[7] Schmidt, D. J.; Cebeci, F. C.; Kalcioglu, Z. I.; Wyman, S. G.; Ortiz, C.; Van Vliet, K. J.; Hammond, P. T. “Electrochemically controlled swelling and mechanical properties of a polymer nanocomposite”, ACS Nano 2009, 3, 2207-2216.
[8] Schmidt, D. J.; Moskowitz, J. S.; Hammond, P. T. “Electrically triggered release of a small molecule drug from a polyelectrolyte multilayer coating”, Chem. Mater. 2010, 22, 6416-6425.
[9] Wang, F.; Liu, X.; Li, G.; Li, D.; Dong, S. “Selective electrodissolution of inorganic ions/DNA multilayer film for tunable DNA release”, Journal of Materials Chemistry, 2009. 19(2): p. 286-291.
[10] Wei, J. J., et al., Electric-stimulus-responsive multilayer films based on a cobaltocenium-containing polymer. Polymer Chemistry, 2014. 5(22): p. 6480-6488.

### SUMMARY OF THE INVENTION

The inventors of the technology disclosed herein have developed a novel fabrication approach for manufacturing electrically active nano-patterned polyelectrolyte multilayer (PEM) films that enable triggered or on-demand drug release. The PEM device of the invention is constructed of an electroactive polyelectrolyte and an oppositely charged polyelectrolyte, i.e., one is a negatively charged and the other is a positively charged polyelectrolyte, that are layered one on top of the other, as shown in the embodiment depicted in FIG. 1. In the particular example, the PEM comprises alternating layers of an electroactive polymer such as the negatively charged polyelectrolyte sulfonate-functionalized poly(3,4-ethylenedioxythiophene) (PEDOTS) and a positively charged polyelectrolyte such as the biocompatible poly (ethylene imine) (PEI). The active material, or drug, is disposed or embedded between the layers.

Application of positive voltage to the PEM constructed according to the invention causes oxidation of the electroactive polymer PEDOTS, rendering the PEDOT backbone positively charged. The reduced electrostatic attraction or repulsion between the oxidized PEDOTS and the positively charged PEI destabilizes the PEM, causes the PEM to disassemble and release the drug contained therein.

In a first aspect, the invention contemplates a nano-sized polyelectrolyte multilayer (PEM) comprising at least one layer of an electroactive polyelectrolyte polymer, the PEM being configured to hold or receive a biologically active material to be disposed within said multilayer and to release said active material under electric field. In some embodiments, the PEM comprises said biological active material.

In some embodiments, the PEM is a stacked nano-structure comprising a plurality of stacked layers, each of said layers being of a polyelectrolyte polymer, wherein at least one of said layers is of an electroactive polymer (being itself a polyelectrolyte polymer).

In some embodiments, the PEM is a stacked nano-structure comprising a plurality of alternating layers of a positively charged polyelectrolyte polymer and a negatively charged polyelectrolyte polymer.

In some embodiments, the PEM is nano-patterned, as define herein.

The invention further provides a nano-patterned (or a nano-sized) polyelectrolyte multilayer (PEM) comprising a plurality of stacked layers, at least one of said plurality of stacked layers is a layer of an electroactive polymer and at least one another of said plurality of stacked layers is a layer of a charged polyelectrolyte polymer, the PEM being configured to hold or receive at least one active material and to release said at least one active material under electric field. In some embodiments, the PEM is provided with at least one active material that is disposed therein.

The invention also provides a nano-patterned (or nano-sized) PEM constructed of alternating stacked layers of an electroactive polymer and of a charged polyelectrolyte polymer, wherein at least one interface between a layer of the electroactive polymer and a charged polyelectrolyte polymer is configured to comprise or receive at least one active material. The PEM is configured and operable to release the at least one active material under electric field. In some embodiments, the interface comprises at least one active material.

The invention further provides a (patterned) device comprising
a surface region having alternating charged and neutral domains, or spaced-apart charged domains surrounded by neutral domains; and
a polyelectrolyte multilayer (PEM) formed on at least one of the charged domains.

In some embodiments, the PEM comprising at least one layer of an electroactive polyelectrolyte polymer, the PEM being configured to hold or receive an active material to be disposed within said multilayer and to release said active material under electric field.

In some embodiments, in a device of the invention, the charged domains are spaced-apart and arranged in any shape or pattern. In some embodiments, the charged domains are linearly patterned and are optionally arranged parallel to each other. In some embodiments, the charged domains are in a shape of a circle or are dot domains surrounded or separated by neutral domains.

In some embodiments, the charged domains are positively charged or negatively charged. In some embodiments, the charged domains are positively charged, surrounded by neutral domains.

The charged domains of the substrate may be formed by nano-patterning the substrate or by causing a substrate material adopt a charge at nano-confined regions. Thus, the term “nano-patterning” stands to mean forming a charged domain, a region or a pattern on a substrate or a substrate material, wherein the charged domain, region or pattern is separated from one or more such other charged domains, regions or patterns by a neutral domain, region or pattern. The charged domain, region or pattern is confined in size or dimension to the nanometric regime, thus being nano-confined and dictating the size of the PEM that may be fabricated thereon. In other words, the charged domain has at least one dimension, e.g., width or thickness, that is in the nanometric regime. Other dimensions may be in the micrometer regime. Where the charged domains are line domains or rectangular domains, the shortest axis of the domain, i.e., its width, has a nanometric size. The length of the rectangular domain may be in the micrometric regime. Similarly, where the domain is a line domain or a rectangular domain, its thickness may also be nanometric.

Where the domain is a point or dot domain or a circular domain, the diameter of the domain may also be nanometric, as defined.

Nano-patterning may be achieved by any means known in the art. Non-limiting examples of nano-patterning techniques include electron beam lithography, nanoimprint lithography, dip-pen lithography, microcontact printing or soft lithography and others. Each of the aforementioned techniques is known to a person versed in the art.

In some embodiments, nano-patterning is achieved by using a block copolymer substrate. Suitable block copolymers include such which have a neutral component and a reactive component which is susceptible of adopting a charge or undergoing a chemical transformation into a charged form. The reactive component may be a pH-sensitive group; a hydrolysable group; a photo-reactive group (capable of undergoing dissociation or structural change to yield a charged functionality); a thermosensitive group; a tertiary amine group (capable of undergoing quaternization); an acidic group (capable of ionization, e.g., carboxylic acids, sulfonic acids, sulfinic acids); an ester group or an amide group or an anhydride group or any such group capable of hydrolysis to yield a charged functionality, e.g., a carboxylate group or an hydroxide group; a sulfur group; an oxygen group (capable of ionization to yield a positive or a negative charge); and others.

In some embodiments, the block copolymer is selected to have an amine group, optionally a tertiary amine, capable or undergoing quaternization.

In some embodiments, the block copolymer has a block component selected from neutral or inert blocks such as polyisoprene, polyethylene oxide (PEO), polypropylene oxide (PPO), polystyrene, poly(methyl methacrylate), polybutadiene and others. The copolymer component may be selected to comprise one or more charged functionalities or such functionalities capable of adopting a charge, as disclosed herein.

Non-limiting examples of block copolymers that can be used as substrate materials in accordance with the invention include polystyrene-block-poly (2-vinyl pyridine) (PS-b-P2VP), polystyrene-block-poly(acrylic acid), polystyrene-block-poly(tert-butyl acrylate), polystyrene-block-poly(N-acrylamide), polystyrene-block-poly(lactic acid) and others.

In some embodiments, the block copolymer is polystyrene-block-poly (2-vinyl pyridine) (PS-b-P2VP). In this exemplary case, the 2-vinyl pyridine (P2VP) domains of the PS-b-P2VP copolymer may be crosslinked by reaction of the pyridine units with a crosslinking agent, e.g., 1,4-diiodobutane (DIB). The crosslinking causes quaternization of the pyridine units and renders the crosslinked P2VP domains (denoted herein “xP2VP”) positively charged, while maintaining the polystyrene blocks (PS) neutral. With the P2VP domains now positively charged, the copolymer substrate (denoted herein “xBCP”) can serve as a template for the preparation of the nano-patterned PEMs.

Other copolymers may be reacted in a similar fashion to achieve formation of charged domains (negatively charged or positively charged and neutral domains).

A patterning method is disclosed, for example, in Soft Matter 2016, 12, 4595; Polymer 2016, 107, 406; Soft Matter 2016, 12, 8098; Polymer 2017, 126, 56, each of which being herein incorporated by reference.

In some embodiments, the surface domains are not particles, e.g., nanoparticles. In some embodiments, the substrate is not a particle, e.g., a nanoparticle.

The surface domains (charged and neutral) or the PEMs formed thereon are said to be nano-sized. Each of the PEMs layers is composed of a polyelectrolyte polymer, as disclosed herein, and has at least one dimension (length, width and thickness) in the nanometric regime. Also, each of the charged and neutral domains (length, width or diameter) of the substrate has at least one dimension in the nanometric regime.

Each of the charged domains or at least one thereof, is nano-patterned to have a width that is a few nanometers in size. In some embodiments, each of the domains or at least one thereof is nano-patterned to a width in the range of 5 and 150 nm.

In some embodiments, the width is in the range of 1 and 150, 1 and 140, 1 and 130, 1 and 120, 1 and 110, 1 and 100, 1 and 90, 1 and 80, 1 and 70, 1 and 60, 1 and 50, 1 and 40, 1 and 30, 1 and 20, 1 and 10, 1 and 5, 5 and 150, 5 and 140, 5 and 130, 5 and 120, 5 and 110, 5 and 100, 5 and 90, 5 and 80, 5 and 70, 5 and 60, 5 and 50, 5 and 40, 5 and 30, 5 and 20, 5 and 10, 10 and 150, 10 and 140, 10 and 130, 10 and 120, 10 and 110, 10 and 100, 10 and 90, 10 and 80, 10 and 70, 10 and 60, 10 and 50, 10 and 40, 10 and 30 or between 10 and 20 nm. In some embodiments, the width is between 10 and 150 nm. In some embodiments, the width is between 1 and 10 nm.

In some embodiments, each of the PEM's layers may have a thickness between 0.1 nm and 50 nm. The thickness may be between 0.1 and 50, 0.2 and 50, 0.3 and 50, 0.4 and 50, 0.5 and 50, 0.6 and 50, 0.7 and 50, 0.8 and 50, 0.9 and 50, 1 and 50, 2 and 50, 3 and 50, 4 and 50, 5 and 50, 6 and 50, 7 and 50, 8 and 50, 9 and 50, 10 and 50, 15 and 50, 20 and 50, 25 and 50, 30 and 50, 35 and 50, 40 and 50, 0.1 and 10, 0.2 and 10, 0.3 and 10, 0.4 and 10, 0.5 and 10, 0.6 and 10, 0.7 and 10, 0.8 and 10, 0.9 and 10, 1 and 10, 2 and 10, 3 and 10, 4 and 10, 5 and 10, 6 and 10, 7 and 10, 8 and 10, 9 and 10, 0.1 and 5, 0.2 and 5, 0.3 and 5, 0.4 and 5, 0.5 and 5, 0.6 and 5, 0.7 and 5, 0.8 and 5, 0.9 and 5, 1 and 5, 2 and 5, 3 and 5 or 4 and 5 nm.

In general, the thickness (height) of the entire PEM multilayer is not restricted, and could span several nanometers and several millimeters in thickness. In some embodiments, the thickness of the PEM is between 5 nm and 5 microns. In some embodiments, the thickness of the PEM is in the nano regime, i.e., being below 1000 nm or below 900 nm or below 800 nm or below 700 nm or below 600 nm or below 500 nm or below 400 nm or below 300 nm. In some embodiments, the thickness of the PEM is between 5 and 300, 5 and 290, 5 and 280, 5 and 270, 5 and 260, 5 and 250, 5 and 240, 5 and 230, 5 and 220, 5 and 210, 5 and 200, 5 and 190, 5 and 180, 5 and 170, 5 and 160, 5 and 150, 5 and 140, 5 and 130, 5 and 120, 5 and 110, 5 and 100, 5 and 90, 5 and 80, 5 and 70, 5 and 60, 5 and 50, 5 and 40, 5 and 30, 5 and 20, 5 and 10, 10 and 300, 10 and 290, 10 and 280, 10 and 270, 10 and 260, 10 and 250, 10 and 240, 10 and 230, 10 and 220, 10 and 210, 10 and 200, 10 and 190, 10 and 180, 10 and 170, 10 and 160, 10 and 150, 10 and 140, 10 and 130, 10 and 120, 10 and 110, 10 and 100, 10 and 90, 10 and 80, 10 and 70, 10 and 60, 10 and 50, 10 and 40, 10 and 30 or between 10 and 20 nm.

In some embodiments, the thickness of the PEM is between 500 nm and 5 microns. In some embodiments, the thickness is between 500 nm and 3 microns, 500 nm and 2.5 microns, 500 nm and 2 microns, 500 nm and 1.5 microns, 500 nm and 1 micron, 1 and 5 microns, 1 and 4.5 microns, 1 and 4 microns, 1 and 3.5 microns, 1 and 3 microns, 1 and 2.5 microns, or 1 and 1.5 microns.

In some embodiments, the PEM thickness is between 5 nm to above 5 microns, wherein each layer having a thickness of 0.1 nm to a 50 nm.

In some embodiments, the length of the charged domains, being in some embodiments rectangular in shape, is between several microns and several millimeters.

As stated herein, a PEM of the invention formed on a charged surface domain comprises a plurality of alternating positively and negatively charged polyelectrolyte polymer layers, wherein the polymer of at least one of the charged polyelectrolyte polymer layers is an electroactive polyelectrolyte polymer. As used herein, the “polyelectrolyte polymer” is a polymer that is composed of charged monomer subunits, which endow the polymer either positively or negatively charged. While the charged monomer subunits may be identical in structure, composition and charge, in some embodiments, these subunits may not be identical. Irrespectively, the polymer is either positively or negatively charged. The “electroactive polymer” is similarly a polyelectrolyte polymer that is additionally capable of exhibiting a change in oxidation and thus a change in its charge when stimulated by an electric field.

The polyelectrolyte polymers may be synthetic, naturally occurring or semi-synthetic. The polyelectrolyte polymers used in the present invention may be presented in a variety of molecular architectures. These include linear, branched, comb-like, dendritic or star; may be in a form of homopolymers comprising one type of a repeating unit; random copolymers comprising random sequences of two or more different repeating units, where one or more of these units may be charged; block copolymers comprising two or more blocks of homopolymers joined together, where one or more of these blocks may be charged.

The polyelectrolyte polymer is typically not provided in the form of particles or nanoparticles. The polyelectrolyte materials used in accordance with the invention are all polymeric materials. Non-polymeric polyelectrolyte materials are excluded and do not form part of PEMs of the invention. In other words, PEMs of the invention make use of polyelectrolytes consisting polyelectrolyte polymers. Specifically excluded polyelectrolyte material is Prussian Blue. Similarly, PEMs of the invention are typically free of particles of any type, including nanoparticles, liposomes and vesicles.

The polyelectrolyte polymers may be selected amongst negatively charged polyelectrolytes and positively charged polyelectrolytes.

Non-limiting examples of negatively charged polyelectrolyte polymers include sulfonate-functionalized poly(3,4-ethylenedioxythiophene) (PEDOTS), sulfonate-functionalized poly(styrenesulfonic acid) (PSS), poly(2-acrylamido-2-methyl-1-propane sulfonic acid) (PAMPS), sulfonate-functionalized poly(ether ether ketone) (SPEEK), sulfonate-functionalized lignin, poly(ethylenesulfonic acid), poly(methacryloxyethylsulfonic acid), and others, each of the aforementioned optionally provided in their salt forms, or in any of the architectures described herein, e.g., as copolymers.

Non-limiting examples of positively charged polyelectrolyte polymers include poly(ethylene imine) (PEI), poly(diallyldimethylammonium chloride) (PDAD) and copolymer thereof with polyacrylamide (PDAD-co-PAC), poly(vinylbenzyltrimethylammonium) (PVBTA), ionenes, poly(acryloxyethyltrimethyl ammonium chloride), poly(methacryloxy (2-hydroxy) propyltrimethyl ammonium chloride), and copolymers of any of the aforementioned; polyelectrolytes comprising pyridinium groups, e.g., poly(N-methylvinylpyridine) (PMVP), poly(N-alkylvinylpyridines) and copolymers thereof; protonated polyamines, e.g., poly(allylaminehydrochloride) (PAH), and others.

In some embodiments, the negatively charged polyelectrolyte polymer is sulfonate-functionalized poly(3,4-ethylenedioxythiophene) (PEDOTS) and the positively charged polyelectrolyte polymer is poly(ethylene imine) (PEI).

As defined herein, the electroactive polymer is a polyelectrolyte polymer that may be negatively or positively charged that is additionally capable of exhibiting a change in its charge upon stimulation with an electric field. Non-limiting examples of electroactive polymers include sulfonate-functionalized poly(3,4-ethylenedioxythiophene) (PEDOT), carboxylated or sulfonated derivatives of PEDOT, poly(3-hexylthiophene-2,5-diyl) (P3HT), poly(phenylene vinylene) (PPV), polyacetylene, and others.

As would be understood, a PEM's first layer formed on a charged domain surface would be of a polymer that is opposite in charge to the charge of the domain. In other words, where the charged domain is positively charged, the PEM's first layer is negatively charged, and where the charged domain is negatively charged, the PEM's first layer is positively charged.

Thus, in some embodiments, the surface domain is formed of polystyrene-block-poly (2-vinyl pyridine) (PS-b-P2VP), wherein the charged domain is positively charged; the first PEM layer is formed of a negatively charged polyelectrolyte, as defined herein, being optionally sulfonate-functionalized poly(3,4-ethylenedioxythiophene) (PEDOTS); and the further PEM layer is formed of a positively charged polyelectrolyte, as defined herein, being optionally poly(ethylene imine) (PEI).

In some embodiments, the PEM is structured of alternating PEDOTS and PEI layers, wherein PEDOTS functions as an electroactive polymer.

As discussed and demonstrated herein, application of a positive voltage to a PEM constructed of a negatively charged electroactive polymer, such as PEDOTS, and a positively charged polyelectrolyte polymer, such as PEI, causes oxidation of the negatively charged electroactive polymer, rendering it positively charged or less negatively charged. The electrostatic repulsion or weakened interaction between the transformed positively charged PEDOTS and the positively charged PEI destabilize the PEM, causing it to disassemble (separation of layers). Materials contained in spaces formed between the stacked layers leach out or become released due to the mechanical disassembly or destabilization of the stack.

Devices or PEMs of the invention may be provided free of an active material, which may be added to the device or PEM after its construction, or may be provided with an active material, as disclosed. Bare devices or PEMs are nevertheless constructed and configured to comprise, hold or receive an active material.

Thus, PEMs of the invention may be used as drug delivery vehicles or as on-demand vehicles for release of drug agents. In accordance with the invention, one or more active materials or drug agents or biologically active materials may be contained, hosted or held between layers of the polyelectrolyte polymer in PEMs of the invention. The active material is physically held between the polymer layers, exhibiting low or minimal chemical association with the materials making up the layers. In the absence of e.g., a positive voltage, the active does not become released from the PEM.

The active material, drug agent or “biologically active material”, or any lingual variation thereof, is a material having an observable or measurable biological effect on living cells, bacterial cells and viruses. The active material may be a biological molecule, e.g., amino acid based, nucleic acid based or a small molecule (low molecular weight molecule) having a therapeutic effect. The active material is a material that induces a biological effect by penetrating the cell, wherein cell penetration occurs without utilizing any transport techniques (either associated with the active material or associated with the cells such as receptors and transporters) or agents, within a short period of time and in high concentrations, or an agent that induces an effect by interacting with the cell membrane, for example an agonist/antagonist of a receptor working on the external membrane of the cell, ligands of membrane transporters, agents that promote cellular phagocytosis and the like. Non-limiting examples of such active materials include small chemical molecules (drugs, therapeutic agents and imaging agents), neurotransmitters, hormones, growth factors, amino acid-based molecules (peptides, proteins, lectins, antibodies, enzymes or their fragments), nucleic acid-based agents (DNA, RNA antisense, iRNA, siRNA, microRNA, ribozymes etc.) and combinations thereof. The combination may be of two or more materials of the same type (e.g., two or more peptides) or of two or more different types of materials such as a combination of the Cas9 enzyme and a guide RNA needed to perform the CRISPR-Cas9 technology.

The active may be a neutral active material or a charged active material. The active may be water-soluble or water-insoluble.

When a PEM structure is disassembled in the presence of an electric field, the active(s) contained in the PEM is released from the PEM and floods the device environment. A biological or a therapeutic effect may be achieved by having the device placed next to cells or tissues to which the active is to be delivered. The effect can be achieved by having the active present at high concentration at the vicinity of the cells. The cells are living cells derived from a mammalian source or a non-mammalian source. The cells may be mammalian, non-mammalian eukaryotic cells, prokaryotic cells such as bacteria or plant cells, yeast and fungi cells. In some embodiments, the cells include bacteria and viruses.

Non-limiting examples of cells include human embryonic kidney cells (HEK293), primary cells, non-dividing cells, and difficult-to-transfect cells, such as Jurkat cells.

The cells may also be or include stem cells.

The cells may be present as a single cell layer, as individual cells in aggregates or clusters, or as tissues that contain such cells.

Thus, the invention also provides a device comprising a PEM of the invention, wherein the PEM is associated or in contact with a cell or present in a cell sample.

The invention further provides a method for delivering an active, or a biologically active material to a vicinity of living cells (without substantially reducing cell viability), the method comprising applying a voltage to a PEM positioned in contact with or in proximity of the living cells, the voltage being of a magnitude and applied for a duration sufficient to disassemble the PEM from the substrate on which it is present or disassemble of the PEM multilayer (separation of layers), thereby causing release of the active material into the vicinity of the living cells.

In some embodiments, the method comprises
contacting the living cells with a PEM of the invention (or contacting a PEM of the invention with living cells);
applying a voltage to the PEM, the voltage being of a magnitude and for a duration of time sufficient to disassemble the PEM and release the active material into the vicinity of the living cells.

The invention also provides a method for delivering an active material through a membrane of a living cell (without substantially reducing cell viability), the method comprising applying a voltage to a PEM positioned in contact with or in vicinity of the living cell, the voltage being of a magnitude and applied for a duration sufficient to disassemble the PEM, cause release of the active material and cause a transient change to the membrane of the living cell, thereby enabling penetration of the active material through the cell's membrane.

In some embodiments, the method comprises
contacting the living cell with a PEM of the invention or contacting the PEM with the living cell; and
applying a voltage to a PEM positioned in contact with the living cell, the voltage being of a magnitude and duration sufficient to disassemble the PEM, cause release of the active material and cause a transient change to the membrane of the living cell, thereby enabling penetration of the active material through the cell's membrane (into the cell cytoplasm).

As used herein, the term “contacting” refers to the action of having the living cells in intimate contact with the PEM surface. Contacting may involve adhering or associating the cells onto or with the surface of the PEM and culturing the cells on the device, e.g., for 2 days, in a culture medium. The cells may be present as individual adhered cells, as an adhered continuous layer of cells, in a form of cellular aggregates, in cellular organoids or clusters. As demonstrated, cells adhere to the PEM surface spontaneously.

In some embodiments, the cells need not be in complete and direct contact with the PEM. The cells may be in ‘close proximity’, namely in the vicinity of the PEM. In other words, the cells may be at a distance ranging from direct contact with the PEM to several microns from the PEM surface. In some embodiments, the cells are several nanometers away from the PEM surface to no more than 1-2 microns away.

To cause disassembly of the PEM a voltage is applied to the PEM that is in contact with or in proximity of the living cells, the voltage is of a magnitude and applied for a duration of time sufficient to meet two functions (or sufficient to cause): (1) disassembly of the PEM, and, in some embodiments (2) cause a transient change in the cells membrane, to permit penetration or transport of the active released from the PEM through the cell's membrane. Methods of the invention do not involve any step of causing membrane porosity or imposing a structural change to the cell membrane by chemical agents or application of radiation, ultrasound, etc, or by any other means other than the step of applying voltage, as disclosed herein. Thus, methods known in the art for active delivery of drugs do not make part of methods of the present invention.

The transient change in the cell membrane that enables penetration or transport of the active through the membrane may be a result of electroporation, namely formation of transient pores in the membrane, or transient opening of voltage gate channels such as those appearing in neurons, muscle cells, cardiomyocytes and secreting cells. The transient change may also be transient disruption of the ordered bilayer structure of the membrane enabling penetration of the active therethrough. The transient change in the cellular properties does not damage the viability of the cells to any measurable extent. While typical electroporation methods require application of a high voltage in the range of 1,000-1,500V, a method of the invention can cause penetration of actives into the cells by application of a voltage that is substantially low. The voltage may be below 50V, or below 40V, or below 30V, or below 20V, or below 10V, or below 5V or below 4V, or below 3V, or below 2V. In some embodiments, the applied voltage is between 1 and 50V, or between 1 and 20V, or between 1 and 10V, or between 1 and 5V, or between 1 and 2V, at room temperature (23-30° C.). Voltage is applied where the cells and the PEM is immersed or present in a medium comprising or consisting a buffer solution.

Voltage may be applied for a period of several milliseconds (e.g., 1 millisecond), seconds or several minutes (e.g., up to 30 minutes).

The invention further provides a method for on-demand release of an active material from a PEM of the invention, the active material being released in a vicinity of viable cells to induce an effect on the cells membrane or induce penetration or transport of the actives into the cells, as disclosed herein.

Methods of the invention can be used for clinical purposes, for safely and efficiently transport materials into cells without damaging their viability. In an exemplary use, cells extracted from a subject are treated ex vivo, e.g., for manipulating genetic materials, and are thereafter re-introduced to the subject. A non-limiting example of such a use is CAR-T manipulation of T-cells and in CRISPR-Cas9 manipulation.

Methods of the invention may also be used in research. Some examples include:

Stem cell research: The innovation can allow the transfection of primary cells, non-dividing cells, and difficult-to-transfect cells, such as Jurkat cells. Some of the aforementioned cells may be important for studying T cell leukemia and T cell signaling. It can therefore advance the discovery of new cancer treatments and drugs for autoimmune diseases.

Pharmaceutics: an integral part of today's drug discovery procedure is the testing of new drugs on immortalized cell lines, which serve as models for complex biological systems. However, only a limited variety of cell lines are available—the ability to genetically engineer them would enable to create novel cell lines that will alleviate research on drugs. Transfecting these cells using low voltage would lead to the establishment of a high efficiency method for gene editing with CRISPR that causes minimal damage to the cells, making CRISPR a much more reliable and efficient toolkit. It would allow generating tissues ex vivo for transplantation.

Biochemistry: using cell lines is an economic way of growing in vitro cells that are similar to those found in a multicellular organism. These cells are widely used for testing toxicity of compounds and drugs as well as producing eukaryotic proteins. Genetically engineered cell lines would considerably expand the variety of cells available for these studies.

Methods of the invention can also be used in forming engineered plants.

Devices of the invention may be used applications such as biosensors, coatings with antibacterial or antifouling properties; may be used in drug delivery, as functional films that can release drugs an electric field.

PEMs of the invention may comprise anywhere from 2 and 50 layers. In some embodiments, the number of layers is between 5 and 30, 5 and 20, 5 and 10, 10 and 30 or 10 and 20.

The invention further provides a method for producing a PEM or device according to the invention, e.g., a PEM holding an active material, the method comprising constructing on a charged region of a nano-patterned substrate having a charged region and a neutral region a stacked plurality of layers of charged polyelectrolyte polymers, wherein each layer having a charge opposite to a charge of a preceding layer and also opposite to a charge of a subsequent layer, a first layer of the plurality of layers formed on a surface of the charged region having a charge opposite to the charge of the charged region, and wherein the stacked layers are formed layer by layer (LbL, bottom up).

In some embodiments, the LbL construction of the device is conducted under low voltage. The low voltage used for the fabrication of the device does not affect its structural integrity, i.e., is not sufficient to cause its disassembly.

In some embodiments, the method comprises deposition of at least one active material. In some embodiments, the active material is deposited on a negatively charged layer prior to deposition thereon of a positively charged layer, and vice versa.

In some embodiments, the active material is deposited so that at least one interface between a negatively charged layer and a positively charged layer comprises the active material.

In some embodiments, the method comprising

(a) on a nano-patterned substrate having a charged region and a neutral region, forming a first layer of a first charged polyelectrolyte polymer, the first charged polyelectrolyte polymer having a charge opposite to the charge of the charged region;

(b) on the first layer forming a second layer of a second charged polyelectrolyte material, the second charged polyelectrolyte polymer having a charge opposite to the charge of the first charged electrolyte polymer;

(c) on the second layer forming a further layer of a further charged polyelectrolyte polymer, the further charged polyelectrolyte polymer having a charge opposite to the charge of the charged electrolyte polymer of the underlying layer; and

(d) repeating steps (b) and (c) one or more times to construct the PEM.

In some embodiments, at least one active material is deposited on one layer and subsequently covered by deposition of another layer of the opposite charge. In some embodiments, the active material may be deposited only after a certain number of layers have been deposited.

As noted herein, at least one of the polyelectrolyte polymers deposited is an electroactive polyelectrolyte polymer.

In some embodiments, all negatively charged polymer layers are of the same polymeric material and all positively charged polymer layers are of the same polymeric material. In some embodiments, the PEM may be a hybrid structure comprising layers of different positively charged polymeric materials (two or more) or layers of different negatively charged polymeric materials (two or more).

In some embodiments, the method comprises obtaining a nano-patterned substrate having a charged region and a neutral region. In some embodiments, the nano-patterned substrate is formed of a block copolymer, as disclosed herein. In some embodiments, the nano-patterned substrate is formed of a block copolymer having a neutral component and a reactive component which is susceptible of adopting a charge or undergoing a chemical transformation into a charged form, as disclosed herein.

In some embodiments, the charged region of the nano-patterned substrate is positively charged and the first charged polyelectrolyte polymer is negatively charged, and selected as herein.

In a PEM device of the invention, each layer is of a charge opposite to a charge of the neighboring layers. Thus, a first layer is of a negatively charged polyelectrolyte polymer, the second layer is of a positively charged polyelectrolyte polymer, the further layer is of a negatively charged polyelectrolyte polymer and so forth, wherein all layers composed of a positively charged polymer are of the same material and all layers composed of a negatively charged material are of the same polymer.

In some embodiments, each layer is formed by dipping the device in a solution of the polymer to be deposited as a layer. For example, for forming the first layer, the substrate is dipped in a solution of a polyelectrolyte of a charge opposite to that of the charged domains of the substrate.

Each layer of a PEM of the invention is characterized by a surface roughness on the order of a few nanometers. The roughness facilitates kinetic trapping of active materials, which may be neutral, charged or water soluble. In some embodiments, deposition, coating or otherwise application of the active material may be by spin coating. The active material need not coat the full surface of the layer. Coating of a part or a portion or a region of the layer is sufficient.

A PEM of the invention may comprise one active material or a plurality of such materials. In some embodiments, each of the active materials is water soluble. In some embodiments, for achieving spin coating an aqueous formulation of the active material(s) is provided.

In some embodiments, the active material is a poly-charged material, such as a DNA. In such cases, the active material may be incorporated into the polyelectrolyte layer itself.

The invention further provides a device comprising
a surface region composed of polystyrene-block-poly (2-vinyl pyridine) (PS-b-P2VP) having alternating positively charged and neutral domains; and
a polyelectrolyte multilayer (PEM) present on at least one of the positively charged domains, the PEM comprising alternating layers of a sulfonate-functionalized poly(3,4-ethylenedioxythiophene) (PEDOTS) and poly(ethylene imine) (PEI), at least one interface between said alternating layers comprising at least one biologically active material, the device being configured and operable to release said at least one biologically active material upon application of an electric field.

Also provided is a method for delivering an active material to a vicinity of a living cell or through a membrane of a living cell, the method comprising applying a voltage to a device of the invention when positioned in contact with or in proximity to the living cell, the voltage being of a magnitude and applied for a duration sufficient to disassemble the PEM, release the active material from the device and cause a transient change to the membrane of the living cell, thereby enabling the material penetration through the cell membrane.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts the components and production scheme of a PEM assembly according to an embodiment of the present invention.

FIGS. 2A-H present SEM images (A,D), SFM height images (B,E) and cross-sections (C,F) of the patterned substrate before (A-C) and after (D-F) deposition of the first PEDOTS layer. SEM images were taken at 26° tilt angle. SFM cross-sections represent averaging over 40 adjacent scan lines (800 nm×150 nm box). (G) XPS data of the block copolymer film (blue), the nano-patterned template (xBCP) formed after reaction with DIB (red), and the film after deposition of the first PEDOTS layer (black). (H) Cell proliferation and viability data on different substrate interfaces.

FIGS. 3A-D present fluorescence images (488 nm excitation) of the PEM assembled on the xP2VP film (A,B) and on the xBCP film (C,D), assembled with (B,D) and without Dox (A,C). The micro-scale pattern appearing in the xBCP images reflects thickness undulations in the film.

FIGS. 4A-F provide (A,B) Quantification of the amount (in nanomole per cm² of film) of stored Dox in the polyelectrolyte films assembled on different substrates (P2VP homopolymer (xP2VP) vs. block copolymer (xBCP)) via its release in a 1 mL PBS using (A) sonication or (B) application of 20 cyclic voltammetry sweeps from −0.2 V to 0.8 V at 0.1 V/s rate. (C,D) Leakage test, performed by incubation of 3.5-bilayer PEMs: (C) amount of Dox released into a 1 mL PBS; (D) amount of Dox remaining in the multilayers, quantified after subsequent sonication into a fresh 1 mL of PBS. (E) Quartz crystal microbalance experiment, showing the buildup of the layers and their disassembly. Numerical labels denote the process steps: (1) introduction of 1 wt % PEI solution; (2) washing with water; (3) introduction of 1 wt % PEDOTS solution; (4) disassembly upon the application of 10 voltage cycles from −0.2 V to 0.8 V at 0.1 V/s rate. Washing cycles following deposition were associated by a slight increase in frequency, owing to the desorption of weakly associating polyelectrolytes. Insets show expanded regions of the disassembly step. (F) Quantification of Dox released from the multilayer assembled on the nano-patterned substrate into 1 mL of deionized water after different number of voltage cycles from −0.2 V to 0.8 V at 0.1 V/s rate.

FIGS. 5A-F provide fluorescence images of a live/dead assay of fibroblast cells cultured for 2 days on PEDOTS/PEI multilayers that were assembled on the block copolymer template: (A) without Dox; (B) without Dox, after 20 cycles of electrochemical stimulation_{;} (C) with Dox, without electrochemical treatment; (D-F) with Dox, after 1, 5, and 10 cycles of electrochemical stimulation.

FIGS. 6A-C provide fluorescence images of a live/dead assay of fibroblast cells cultured for 2 days on PEDOTS/PEI multilayers that were assembled on the xP2VP template: (A) without Dox; (B) without Dox, after 20 cycles of electrochemical stimulation; (C) with Dox, without electrochemical treatment.

FIGS. 7A-D provide (A,B) Overlaid optical microscopy and fluorescence images (λₑₓ=481 nm) showing extent of Dox internalization in NIH3T3 fibroblast cells after 24 and 36 h incubation in 17 μM Dox solution (for comparison, the Dox concentration that was released from 1-cm² nano-patterned PEM to 1-mL solution was ˜14 μM as shown in FIG. 4F). Cells were cultured for 2 days prior to incubation. (C,D) Live/dead assays corresponding to the images shown in (A,B).

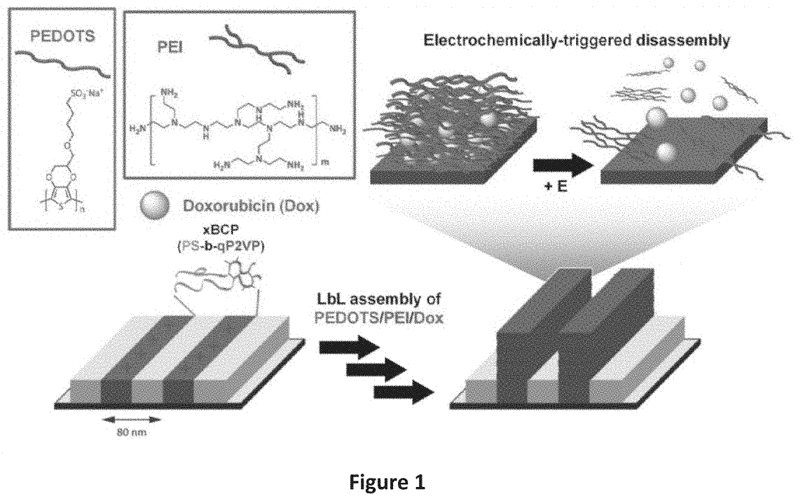

### DETAILED DESCRIPTION OF EMBODIMENTS

### Nano-Patterned Substrate Characterization

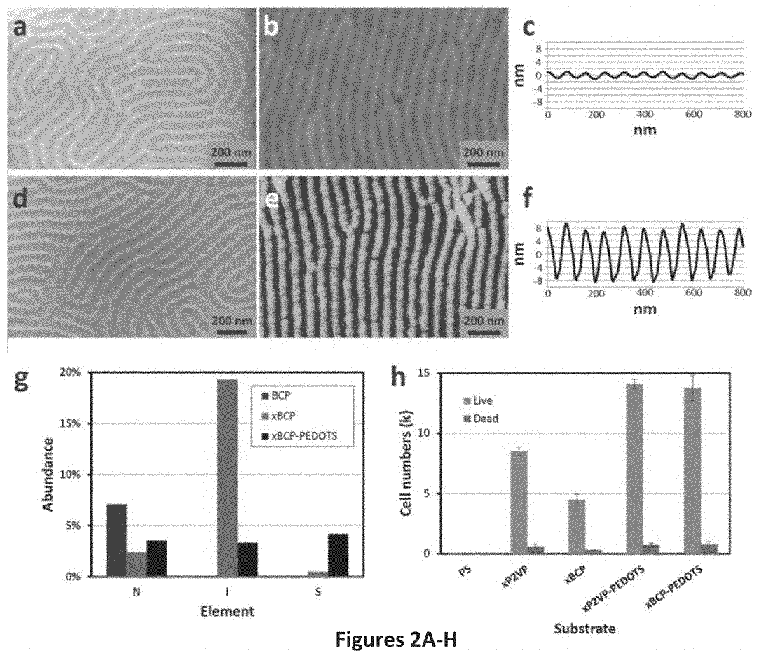

FIGS. 2A-F present the scanning electron microscopy (SEM) and scanning force microscopy (SFM) images of the cross-linked, nano-patterned block copolymer template before and after the deposition of the first PEDOTS layer. A strong increase in height contrast (from ca. 4 nm to ca. 15 nm) indicates that the PEDOTS adsorbed specifically to the positively-charged xP2VP domains. X-ray photoelectron spectroscopy (XPS) measurements performed on the block copolymer film, the crosslinked template (xBCP), and the template after the deposition of the first PEDOTS layer (FIG. 2G) shows a decrease in the intensity of the nitrogen peak after reaction with DIB, which is attributed to the conversion of the surface pyridine groups into alkylated pyridinium ions. Additionally, a strong decrease in the intensity of the iodine peak as well as an evolution of a sulfur peak is noted after PEDOTS deposition, which corroborates the displacement of iodide anions with the PEDOTS during the electrostatic self-assembly process. Lastly, water contact angle measurements show that the PEDOTS layer renders the substrate more hydrophilic (static contact angle decreased from 66.0°±0.3° on the xBCP template to 49°±2° on the xBCP-PEDOTS surface).

Biological testing of cell proliferation and viability were performed on the different substrates (FIG. 2H). Cells did not proliferate on the PS substrate, but adhered nicely on the xP2VP substrate. The amount of cells on the xBCP substrate was about half that on the xP2VP substrate, an intermediate value between PS and xP2VP substrates, reflecting the areal fraction occupied by the xP2VP domains in the xBCP template. However, deposition of PEDOTS increased the number of live cells by about 65% compared to the number of cells cultured on the xP2VP substrate, demonstrating the biocompatibility of PEDOTS. Interestingly, the PEDOTS coating on the xP2VP domains of the xBCP template diminishes the effect of the presence of the PS domains, despite the fact that microscopy data indicates highly selective deposition on the xP2VP domains.

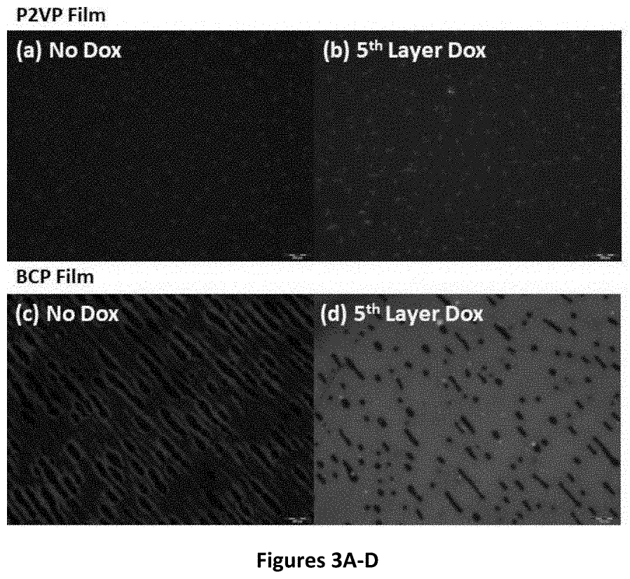

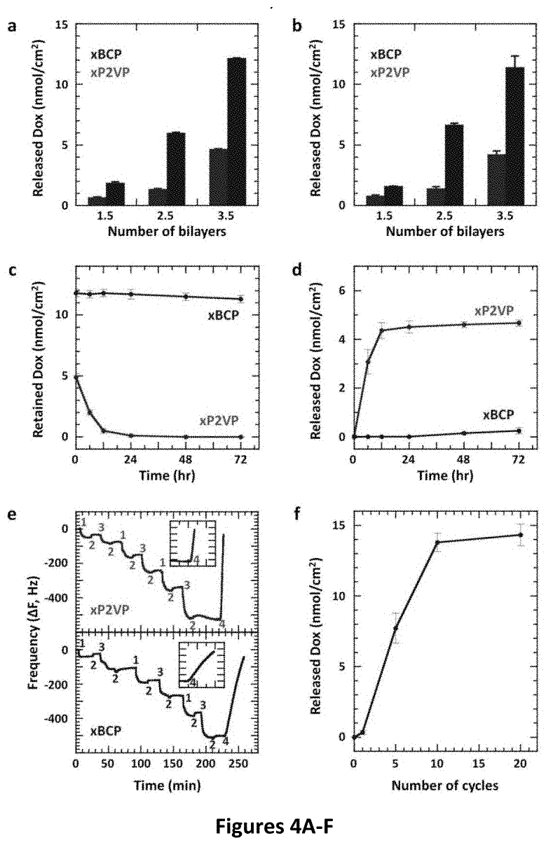

Assembly and quantification of doxorubicin inside the polyelectrolyte multilayers. Polyelectrolyte multilayers consisting of 3.5-bilayers incorporating Dox were assembled on the homogeneous xP2VP and on the nano-patterned xBCP substrates. After the initial PEDOTS layer was deposited, we first tried to absorb the Dox by dipping the substrate into Dox solutions after each polyelectrolyte deposition step. However, only limited amount of Dox was absorbed onto the PEDOTS or PEI top layer. In order to increase the Dox loading within the PEMs, layers of Dox were spin-coated after each polyelectrolyte deposition. Probing Dox fluorescence in the films (λₑₓ=481 nm) shows increased emission from the films after deposition of 3.5 bilayers containing Dox (FIG. 3). Quantification of Dox inside the layers at different stages of the multilayer buildup was performed by releasing Dox into solution using two independent techniques, namely sonication and the application of voltage scans (FIGS. 4A-B; see Experimental Section). Both techniques yielded rather similar values, showing a consistent increase in the amounts of stored Dox with the number of bilayers on both types of substrates. However, the amounts of Dox stored in the nano-patterned multilayers are considerably higher than that in the multilayers assembled on the laterally homogeneous xP2VP substrates.

The observation that the multilayers assembled on the homogeneous xP2VP substrate stored considerably less Dox compared to the amount stored in the nano-patterned xBCP substrate is rather surprising; considering the areas available for polyelectrolyte assembly on both substrates (i.e., the xP2VP domains in the nano-patterned substrates compared to the entire substrate in the xP2VP homopolymer), one would expect the opposite. Moreover, incubation of both 3.5-bilayer Dox-containing films in 1 mL of phosphate buffer saline (PBS; pH 7.5) for extended periods of time revealed that multilayers assembled on the un-patterned homopolymer substrate was considerably less stable in terms of retaining the Dox, which leaked out from the multilayer within ˜12 hours (FIG. 4C,D). In comparison, the multilayer assembled on the xBCP retained its stored Dox for prolonged time, with less than 5% Dox lost after 3 days of continuous incubation (an average leakage rate of 1.6% per day; FIGS. 4C,D). We propose two possible explanations for such a behavior. The first relates to our previous observation that polyelectrolytes deposited over the interface between the PS and xP2VP domains fold back into the xP2VP domains during the drying stage. This possibly helps encapsulating the adsorbed Dox molecules within the PEM and retaining them for prolonged times. The second explanation relates to possible leakage of the Dox from defect points in the PEM caused by dust particles. Whereas a single defect point may, in principle, drain an entire continuous multilayer, only a few domains would be affected by it in a nano-patterned PEM, which consist of isolated domains. Although we cannot provide direct evidence to support these explanations, the ability to store and retain high amounts of bioactive molecules is a clear advantage of nano-patterned devices.

Stimulated release of encapsulated doxorubicin. Direct evidence into the mechanism of release was obtained by quartz crystal microbalance (QCM) experiments performed on a PEDOTS/PEI multilayer assembled on both xP2VP- and xBCP-coated QCM resonator (FIG. 4E). Initially, multilayer buildup is demonstrated by the decrease in resonator frequency every time a new polyelectrolyte solution is injected. Washing cycles resulted in a small increase in the frequency owing to desorption of non-specifically adsorbed polyelectrolytes. Application of 10 voltage cycles (from −0.2 V to 0.8 V at 0.1 V/s rate) caused an abrupt increase in the frequency, reaching the original level, indicating complete disassembly of the multilayer. We observed that the multilayer built on xP2VP disassembled more rapidly compared to that on xBCP, which also supports our previous findings on the increased stability of PEMs on nanopatterns.

FIG. 4F shows the extent of Dox released to solution after different number of cycles of cyclic voltammetry sweeps from −0.2 V to +0.8 V at 0.1 V/s rate. Saturation is reached already after 10 scans, suggesting the complete disassembly of the multilayer and release of all the stored Dox.

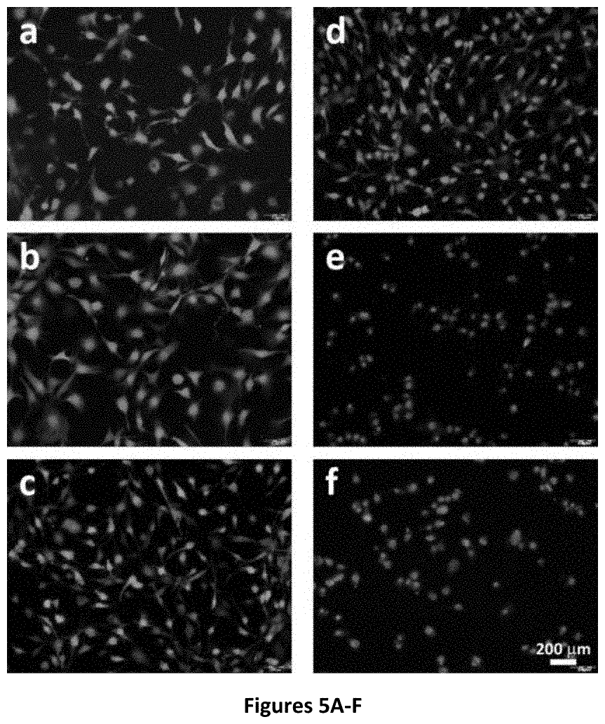

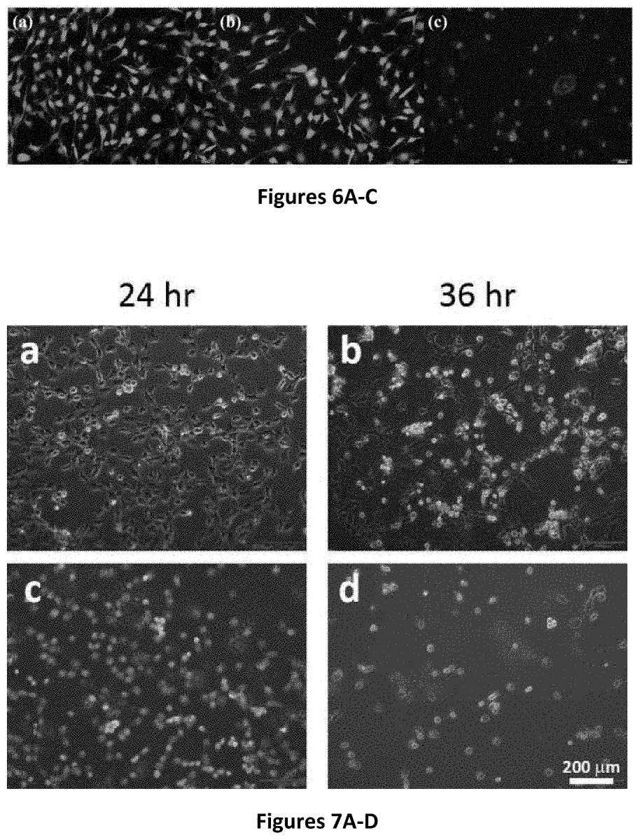

The viability of NIH3T3 fibroblast cells cultured on nano-patterned multilayers were probed (FIG. 5). The cells were cultured for 2 days on the PEM, the multilayer was then subjected to electrochemical treatment, and live/dead assay was performed after 6 additional hours of culturing. FIGS. 5A,B show that cells assembled on nano-patterned PEI/PEDOTS multilayer that did not contain Dox thrive even when voltage scans were applied to the PEM. This indicates that the electrochemical treatment itself does not harm the cells adsorbed on the PEM. Cells adsorbed on a nano-patterned multilayer that contained Dox also thrived as long as no voltage was applied (FIG. 5C), in accord with our previous findings on the ability of the nano-patterned multilayer to retain the stored Dox (FIG. 4C,D). For comparison, cells adsorbed on the Dox-containing PEM assembled on xP2VP did not survive (FIG. 6), owing to Dox leakage from such multilayers (FIGS. 4C,D). Applying a single voltage scan resulted in ˜5% cell death (FIG. 4d); additional scans annihilated the entire population (FIGS. 5E,F). These experiments demonstrated that cell death occurred only because of the triggered release of Dox.

It is noted that the effect of the released drug on the cells is rather quick; much longer time was needed for the drug to penetrate the cell membrane when the cells were incubated with a similar concentration of drug in solution (FIG. 7). This could be attributed to the presence of high local concentrations of the released Dox in the vicinity of the cells, but may also relate to a certain change in membrane permeability induced by the applied voltage.

### CONCLUSIONS

A new platform is disclosed herein that is based on a nano-patterned polyelectrolyte multilayer that enables triggered drug delivery to adsorbed cells. The nano-pattern multilayer is furnished by a hierarchical construction approach, where a microphase-separated block copolymer film serves as a template for the selective deposition of the functional components. The multilayer consists of an electroactive polyelectrolyte, which inverts its charge upon the application of voltage and thus leads to multilayer disassembly and release of an embedded drug.

One of the most interesting and non-trivial attributes of the nano-patterned multilayer is its ability to retain the drug better than the corresponding homogeneous (i.e., non-patterned) multilayer. This ability is explained by the different average conformation of polyelectrolytes when adsorbed on nano-patterned substrates, which may assist in encapsulating the drug in the multilayer, and by the isolation of nano-patterned PEM domains, which reduces leakage from defect points caused by dust particles.

The other important attribute of our system is the relatively high efficacy of drug delivery to cells adsorbed on the surface compared to the delivery efficacy of similar concentration of drug to cells suspended in solution. Two reasons may account for this behavior: a high local concentration of the drug, which is released in close vicinity to the cells, and a possible enhancement in cell permeability caused by the application of voltage.

Overall, we have developed a delivery platform which efficiently encapsulates high loading of biologically active ingredients and controllably releases them upon the application of an external electrical stimulation. Utilizations of this platform technology for cell reprogramming, therapeutic implants and tissue engineering are currently underway.

### EXPERIMENTAL

Preparation of block copolymer templates. Silicon wafers coated with 5 nm titanium adhesion layer and 45 nm gold were pre-cleaned in sulfuric acid-NoChromix (purchased from Sigma-Aldrich) bath overnight and then rinsed with triply distilled water. Thin films of PS, P2VP and PS-b-P2VP were prepared by spin casting from the respective 0.39 wt % chloroform solutions on each substrate at 3000 rpm for 30 s. The films thicknesses (29.5±0.5 nm) were determined by ellipsometry before annealing. The block copolymer films were solvent annealed in a closed petri dish under saturated chloroform vapour for 25 min under ambient conditions. Microphase separation in the BCP films led to the formation of lying P2VP cylinders in PS matrix, which gave rise to surface patterns of alternating stripes of ca. 36 nm width. Films were crosslinked with DIB for 42 hr at 75° C. These conditions led to quaternization of ca. 22% of all pyridine rings (in the volume sampled by an X-ray photoelectron spectroscopy beam) and degree of cross-linking of ˜16%. Fresh films were dipped for 10 min in PEDOTS (10 mM repeat unit concentration, prepared in ultrapure water, 0.055 mSiemens/cm conductivity), rinsed in ultrapure water, and dried by spinning at 2000 rpm for 30 s followed by nitrogen blowing.

Preparation of PEMs on gold electrodes. xP2VP and xBCP films were alternatively immersed in 1 wt % aqueous solutions of PEDOTS and PEI. Each immersion cycle was performed for 15 min and was followed by rinsing with deionized water and then introduction of Dox (by spin coating a 1 mg/mL solution at 2000 rpm for 30 s). The deposition of the last layer (PEDOTS) was followed by drying under nitrogen flow. The fabricated electrodes were stored under 4° C.

Electrochemical setup and disassembly conditions. The electrochemical cell consisted of an Ag/AgCl reference electrode and a Pt counter electrode, and was connected to a potentiostat station (Autolab, Metrohm). The gold electrode coated with Dox-containing 3.5-PEDOTS/PEI bilayer was immersed into PBS (10 mM, pH 7.5) and connected as the working electrode. Cyclic potential sweeps from −0.2 V to +0.8 V were applied at a scan rate 0.1 V/s.

QCM study of multilayer buildup and disassembly. Gold coated QCM sensors were cleaned by RCA-1 procedure (NH₄OH:H₂O₂:H₂O=1:1:4) at 80° C. for 15 min, then rinsed with deionized water and dried under nitrogen flow before use. The cleaned QCM sensors were coated with either P2VP or BCP films, annealed, reacted with DIB, and then coated with the first PEDOTS layer. A coated sensor was mounted into an electrochemical cell and connected as the working electrode; a leakless miniature Ag/AgCl electrode was used as the reference electrode. Polyelectrolyte solutions and deionized water were introduced at a constant flow rate of 50 μL/min. The experiments were started by running deionized water on the chip, and each solution was introduced after the resonance frequency stabilized.

For the disassembly process, the buffer system was first changed to PBS (10 mM, pH 7.5) until a stable frequency measurement was obtained, and then 10 cyclic potential sweeps were applied. The QCM-D signal of the disassembly process was acquired during continuous buffer flow.

Quantification of the amount of stored doxorubicin. Fluorescence images of Dox inside the multilayers were taken using an Olympus BX53 microscope at 488 nm excitation. The amount of Dox stored in the 3.5 bilayer films was quantified by releasing the Dox into 1 mL deionized water using either 30 min sonication or electrochemical scans (see above). The amount of Dox released was quantified using absorption spectroscopy (λₘₐₓ=481 nm; ε=10410 M⁻¹cm⁻¹). Data represent averages of 3 repetitions.

Leakage test. Electrodes coated with 3.5-bilayer films were immersed into 1 mL PBS for 0, 6, 12, 24, 48 and 72 h. Samples were then removed from solution, sonicated in PBS as described above, and the concentrations of Dox in both types of solutions were analyzed by absorption spectroscopy. Data represent averages of 3 repetitions.

Cell culturing. NIH3T3 mice fibroblast cells were cultured for two days on the coated electrodes in the Dulbecco's Modified Eagle Medium (DMEM) supplemented with 10% Fetal Bovine Serum (FBS) at 37° C. under 5% CO₂ atmosphere. Samples were subjected to experiments when cell coverage reached 85-90% confluence.

Cell Viability on coated electrodes. Cell viability before and after multilayer disassembly was performed using live/dead cell double staining kit (purchased from Merck/Sigma-Aldrich), which simultaneously stains viable and dead cells with green and red fluorescence tags, respectively. The stain solutions were added at 37° C. to the cell-covered electrode for 15 min, and images were then taken using a fluorescence microscope (Olympus BX53) with excitation wavelength at 488 nm and 545 nm to differentiate the viable/dead cells. Data represent averages of 3 repetitions.

Triggered drug release and viability assay. Doxorubicin was released from the 3.5-bilayer-coated gold electrode covered with cultured cells using the same conditions described above. After the electrochemical treatment, 2 mL of DMEM were added into the chamber and culturing was continued for additional 6 h at 37° C. under CO₂ atmosphere. The electrodes were then rinsed with PBS three times and stained with live/dead assay kit to determine the amount of viable and dead cells. Data represent averages of 3 repetitions.

### Additional Experimental Details

Instruments. Scanning force microscopy (SFM) images were acquired using a Dimension 3100 scanning probe microscope with a Nanoscope V controller (Veeco, USA). Images were corrected by first-order flattening and processed by the Nanoscope Analysis Program (V1.40, Bruker). BCP height images and film thicknesses were analysed by the built-in depth analysis according to the procedures described elsewhere. High resolution scanning electron microscopy (HR-SEM) images of the films were acquired with a Sirion microscope (FEI Company) at 5 kV acceleration voltage. XPS spectra were recorded on a Kratos ultra axis spectrometer (Kratos Analytical) using mono-energetic Al Kα_{1,2} irradiation (1486.6 eV) with a total power of 144 W (12 kV). ¹H and ¹³C NMR spectra were recorded with Bruker AVA-300 spectrometer and chemical shifts were measured in δ (ppm) with residual solvent peaks as internal standards. Multilayer buildup was monitored on a Qsense quartz crystal microbalance (Biolin Scientific). Absorption spectra were recorded on a Cary 8454 UV-Vis spectrometer equipped with a diode array (Agilent). Film thicknesses measured using a Rudolph monochromatic ellipsometer operating at 633 nm.

Materials. PS-b-P2VP (Mₙ 185 kDa, PDI 1.24, 67 wt % PS, 73 nm lamellar period, as determined by small-angle X-ray scattering) was synthesized by standard anionic polymerization using sec-butyllithium in tetrahydrofuran (THF) under nitrogen atmosphere. The molecular weight, size distribution and polystyrene weight fraction were all determined by gel permeation chromatography (GPC) in tetrahydrofuran (THF) against PS standards for the PS block and comparison of the ¹H NMR signals for phenyl and pyridine groups, respectively, for the P2VP block. P2VP (M_{w} 6.2 kDa, polydispersity 1.04) and PS (M_{w} 9.5 kDa, polydispersity 1.05) were purchased from Polymer Source, Inc. Poly(ethylene imine) (PEI, M_{w} ˜25 kDa by LS, Mₙ ˜10 kDa by GPC) was purchased from Sigma-Aldrich. 1,4-diiodobutane (DIB) and FeCl₃ were purchased from Alfa-Aesar and used as received. 2,3-Dihydrothieno[3,4-b][1,4]dioxin-2-yl methanol (EDOT-OH) and sodium hydride 60% mineral oil suspension were purchased from Angene and Merck/Sigma-Aldrich, respectively. Sodium persulfate and 1,4-butane sulfone were purchased from SHOWA and Acros, respectively. These chemicals were used as received without further purification.

### Synthesis of butanesulfonate-3,4-ethylenedioxythiophene (PEDOTS) Polymer

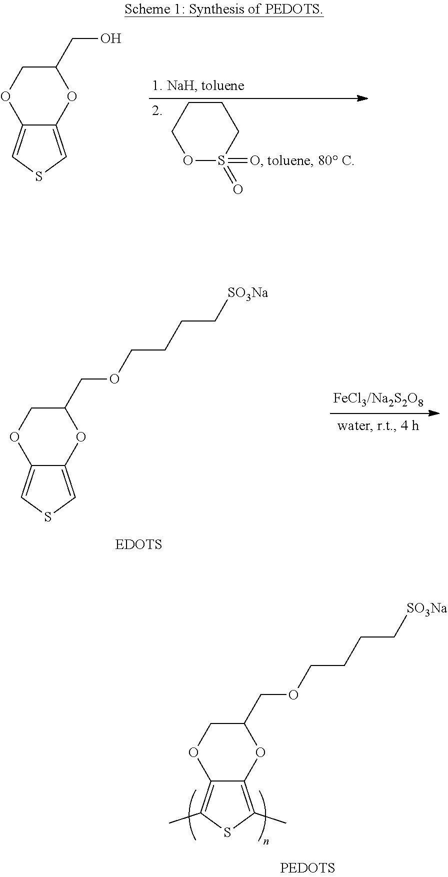

Monomer synthesis. The monomer (4-(2,3-dihydrothieno[3,4-b][1,4]dioxin-2-yl)methoxybutane-1-sulfonic acid sodium salt; EDOTS) was synthesized as previously described.²EDOT-OH (2.5 g, 14.5 mmol) and NaH (0.9 g, 23 mmol) were mixed in a dry 250 mL two-neck round-bottom flask, and dry toluene (50 mL) was added after removing the air by nitrogen blowing. The resulting light brown solution was refluxed at 80° C. for 1.5 h. A solution of butane sulfone (1.98 g, 14.5 mmol) in 15 mL dry toluene was added slowly via syringe after the reaction mixture was cooled to ambient temperature, and then refluxed for another 2.5 h. After cooling to ambient temperature, acetone (150 mL) was poured into the reaction mixture under vigorous stirring. The resulting suspension was filtered, washed with acetone repeatedly and dried to yield light brown powder (4.45 g, 93%). ¹H NMR (300 MHz, D₂O), δ: 6.53 (d, 1H, J=3.6 Hz), 6.51 (d, 1H, J=3.3 Hz), 4.48-4.42 (m, 1H), 4.28 (dd, 1H, J=12.0, 2.1 Hz), 4.11 (dd, 1H, J=11.7, 6.9 Hz), 3.76 (t, 2H, J=4.2 Hz), 3.65-3.60 (m, 2H), 2.94 (t, 2H, J=6.9 Hz), 1.82-1.70 (m, 4H). ¹³C NMR (75 MHz, D₂O) δ: 140.72, 140.57, 100.43, 100.27, 72.78, 71.00, 68.54, 65.64, 50.70, 27.49, 20.87.

Polymer Synthesis

Poly(4-(2,3-dihydrothieno[3,4-b][1,4]dioxin-2-yl)methoxybutane-1-sulfonic acid sodium salt) (PEDOTS, Mₙ 12,000, PDI ˜3) was synthesized according to a literature procedure using FeCl₃/Na₂S₂O₈. EDOTS (0.4 g, 1.21 mmol) was dissolved in 6 mL of DI water. A solution of FeCl₃ (0.01 g, 0.06 mmol) and Na₂S₂O₈ (0.58 g, 2.44 mmol) in water (4 mL) was added dropwise to the above solution and stirred at room temperature (4 h). The reaction mixture was quenched by acetone (50 mL). The precipitated product was centrifuged (5 min, 4000 rpm), separated from the supernatant liquid, re-dissolved in water (10 mL), and precipitated from acetone. This procedure repeated until a clear solution was obtained. Finally, the polymer was dialyzed against deionized water for three days (changing the water every 24 h) using a 1000 g/mol cutoff membrane to yield PEDOTS polymer (51% conversion) after drying.

Molecular weight averages of PEDOTS polymer were determined using a Shimadzu liquid chromatography system (LC-2030C Plus) equipped with a MCX column (Polymer Standards Service, 8×300 mm, 10 μm bead diameter, 10⁵ Å pore size) and an RI detector. The analysis was performed at 23° C. using 0.08 M Na₂HPO₄ aqueous solution as eluent at 0.8 mL/min. The molecular weight values were determined with respect to poly(styrene sulfonate) sodium salt standard kit (Polymer Standards Service) ranging from 1,100-976,000 g/mol.

1. A nano-sized polyelectrolyte multilayer (PEM) comprising at least one layer of an electroactive polyelectrolyte polymer, the PEM being configured to hold or receive an active material to be disposed within said multilayer and to release said active material under electric field.

2. The PEM according to claim 1, being a stacked nano-structure comprising a plurality of alternating layers of a positively charged polyelectrolyte polymer and a negatively charged polyelectrolyte polymer.

3. The PEM according to claim 1, being nano-patterned.

4. The PEM according to claim 1, comprising a plurality of stacked layers, at least one of said plurality of stacked layers is a layer of an electroactive polyelectrolyte polymer and at least one another of said plurality of stacked layers is a layer of a charged polyelectrolyte polymer, the PEM being configured to hold or receive at least one active material and to release said at least one active material under electric field.

5. (canceled)

6. The PEM according to claim 2, wherein the negatively charged polyelectrolyte polymer is selected from sulfonate-functionalized poly(3,4-ethylenedioxythiophene) (PEDOTS), sulfonate-functionalized poly(styrenesulfonic acid) (PSS), poly(2-acrylamido-2-methyl-1-propane sulfonic acid) (PAMPS), sulfonate-functionalized poly(ether ketone) (SPEEK), sulfonate-functionalized lignin, poly(ethylenesulfonic acid), poly(methacryloxyethylsulfonic acid), each of the aforementioned optionally provided in a salt form.

7. The PEM according to claim 2, wherein the positively charged polyelectrolyte polymer is selected from poly(ethylene imine) (PEI), poly(diallyldimethylammonium chloride) (PDAD) and copolymer thereof with polyacrylamide (PDAD-co-PAC), poly(vinylbenzyltrimethylammonium) (PVBTA), ionenes, poly(acryloxyethyltrimethyl ammonium chloride), poly(methacryloxy (2-hydroxy) propyltrimethyl ammonium chloride), and copolymers of any of the aforementioned; polyelectrolytes comprising pyridinium groups; poly(N-methylvinylpyridine) (PMVP), poly(N-alkylvinylpyridines) and copolymers thereof; protonated polyamines; and poly(allylaminehydrochloride) (PAH).

8. The PEM according to claim 1, comprising alternating layers of a sulfonate-functionalized poly(3,4-ethylenedioxythiophene) (PEDOTS) and poly(ethylene imine) (PEI).

9. The PEM according to claim 1, wherein the electroactive polyelectrolyte polymer is a negatively or positively charged polymer exhibiting a change in its charge upon stimulation with an electric field.

10. The PEM according to claim 9, wherein the electroactive polyelectrolyte polymer is a polymer selected from sulfonate-functionalized poly(3,4-ethylenedioxythiophene) (PEDOT), carboxylated or sulfonated derivatives of PEDOT, poly(3-hexylthiophene-2,5-diyl) (P3HT), poly(phenylene vinylene) (PPV) and polyacetylene.

11. The PEM according to claim 1, formed on a charged domain of a substrate having alternating charged and neutral domains.

12. A device comprising
a surface region having alternating charged and neutral domains, or spaced-apart charged domains surrounded by neutral domains; and
a polyelectrolyte multilayer (PEM) formed on at least one of the charged domains, the PEM comprising at least one layer of an electroactive polyelectrolyte polymer, the PEM being configured to hold or receive an active material disposed within said multilayer and to release said active material under electric field.

13. The device according to claim 12, wherein the charged domains are positively charged.

14. The device according to claim 12, wherein the charged domains are formed by nano-patterning the surface region or by causing the surface region material adopt a charge at nano-confined regions.

15. The device according to claim 14, wherein nano-patterning is achievable by using a block copolymer substrate.

16. The device according to claim 15, wherein the block copolymer is polystyrene-block-poly (2-vinyl pyridine) (PS-b-P2VP), polystyrene-block-poly(acrylic acid), polystyrene-block-poly(tert-butyl acrylate), polystyrene-block-poly(N-acrylamide), or polystyrene-block-poly(lactic acid).

17. The device according to claim 12, wherein the active material is a drug, a therapeutic agent, an imaging agent, a neurotransmitter, a hormone, a growth factor, a peptide, a protein, lectin, an antibody, an enzyme, DNA, RNA antisense, iRNA, siRNA, microRNA, a ribozyme and combinations thereof.

18. A method for delivering an active material, or a biologically active material to a vicinity of living cells, or through a membrane of a living cell, the method comprising applying a voltage to a PEM positioned in contact with or in proximity to the living cells, the voltage being of a magnitude and applied for a duration sufficient to disassemble the PEM from the substrate on which it is present or disassemble the PEM multilayer, causing release of the active material into the vicinity of the living cells, thereby enabling penetration of the active material through the cell membrane.

19. (canceled)

20. A device comprising
a surface region composed of polystyrene-block-poly (2-vinyl pyridine) (PS-b-P2VP) having alternating positively charged and neutral domains; and
a polyelectrolyte multilayer (PEM) present on at least one of the positively charged domains, the PEM comprising alternating layers of a sulfonate-functionalized poly(3,4-ethylenedioxythiophene) (PEDOTS) and poly(ethylene imine) (PEI), at least one interface between said alternating layers comprising at least one biologically active material, the device being configured and operable to release said at least one biologically active material upon application of an electric field.

21. The method according to claim 17, for delivering an active material to a vicinity of a living cell or through a membrane of a living cell, the method comprising applying a voltage to the PEM device when positioned in contact with or in proximity to the living cell, the voltage being of a magnitude and applied for a duration sufficient to disassemble the PEM, release the active material from the device and cause a transient change to the membrane of the living cell, thereby enabling the material penetration through the cell membrane.
